(19) United States
(12) Patent Application Publication
(10) Pub. No.: US 2007/0060801 A1
(43) Pub. Date: Mar. 15, 2007

(54) TRANSCUTANEOUS INTRODUCER ASSEMBLY

(75) Inventor: Mark Neinast, Portland, OR (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: ISENSE CORPORATION, Portland, OR (US)

(21) Appl. No.: 11/468,673

(22) Filed: Aug. 30, 2006

Related U.S. Application Data
(60) Provisional application No. 60/712,436, filed on Aug. 31, 2005.

(51) Int. Cl. A61B 5/00

(52) U.S. Cl. 600/300

(57) ABSTRACT

Embodiments of the present invention provide for analyte sensor introducing assemblies. A transcutaneous introducer assembly is provided, comprising a module base adapted for mounting onto an animal's skin, and supporting an electrical network; a handle defining an interior volume; a hollow introducer, having a slot extending at least partially along the length thereof, carried by and extending from the handle in a predetermined orientation, a sensor residing in the hollow introducer prior to and during insertion; and a retraction mechanism adapted to retract and remove the handle and, optionally, the introducer from the module base and the sensor.

### CROSS REFERENCE TO RELATED APPLICATIONS

The present application claims priority to U.S. Patent Application No. 60/712,436, filed Aug. 31, 2005, entitled “Transcutaneous Introducer Assembly Set,” the entire disclosure of which is hereby incorporated by reference in its entirety.

### TECHNICAL FIELD

Embodiments of the present invention relate to the field of analyte sensors, more specifically, to assemblies for introducing analyte sensors.

### BACKGROUND

The insertion of a transcutaneous sensor has proved a difficult problem. In some situations, it is advantageous that a transcutaneous sensor be made as thin as possible so that the disruption of the skin is minimized. With current materials, however, it is difficult for such a thin device to be made strong enough to breach the skin.

For certain applications, a hypodermic needle may be used as an introduction device or trocar (medical breaching device), but if the transcutaneous sensor is threaded through the hypodermic needle, then the removal of the transcutaneous sensor from within the hypodermic needle presents a difficulty. Moreover, the use of a needle introduces the possibility of patient or medical personnel injury by way of the sharp point of the needle.

### BRIEF DESCRIPTION OF THE DRAWINGS

Embodiments of the present invention will be readily understood by the following detailed description in conjunction with the accompanying drawings. To facilitate this description, like reference numerals designate like structural elements. Embodiments of the invention are illustrated by way of example and not by way of limitation in the figures of the accompanying drawings.

FIGS. 1A and 1B illustrate perspective views of an introducer assembly in accordance with various embodiments of the present invention;

FIG. 2 illustrates a cross-sectional view of an introducer assembly in accordance with various embodiments of the present invention;

FIG. 3 illustrates a cross-sectional view of an introducer assembly in accordance with various embodiments of the present invention;

FIG. 4 illustrates a cross-sectional view of an introducer assembly in accordance with various embodiments of the present invention;

FIG. 5 illustrates a perspective view of an introducer assembly in accordance with various embodiments of the present invention; and

FIGS. 6, 7, and 8 illustrate perspective views of introducers in accordance with various embodiments of the present invention.

### DETAILED DESCRIPTION OF EMBODIMENTS OF THE INVENTION

In the following detailed description, reference is made to the accompanying drawings which form a part hereof wherein like numerals designate like parts throughout, and in which is shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the present invention, a phrase in the form “A/B” means A or B. For the purposes of the present invention, a phrase in the form “A and/or B” means “(A), (B), or (A and B)”. For the purposes of the present invention, a phrase in the form “at least one of A, B, and C” means “(A), (B), (C), (A and B), (A and C), (B and C), or (A, B and C)”. For the purposes of the present invention, a phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention provide assemblies for introducing analyte sensors into a body. In an embodiment, there is provided a transcutaneous introducer assembly, comprising a module base adapted for mounting onto an animal's skin (for example mammalian skin, such as human skin), and supporting an electrical network; an elongated handle defining an interior volume; a hollow introducer, such as a needle, having a slot extending at least partially along the length thereof, carried by and extending from the handle in a predetermined orientation relative to and protruding from the module base when the handle and base are coupled, a sensor residing in the hollow introducer prior to and during insertion; and a retraction mechanism adapted to retract and remove the handle and, optionally, the introducer from the module base and the sensor.

In embodiments, a sensor introduced according to an embodiment of the present invention may be rigid or flexible. In an embodiment, a flexible sensor is one that may be flexed repeatedly, such as the type of flexion experienced by a subcutaneously implanted sensor in a human during normal movement, over a period of time (such as 3-7 days or more) without fracture. In an embodiment, a flexible sensor may be flexed hundreds or thousands of times without fracture.

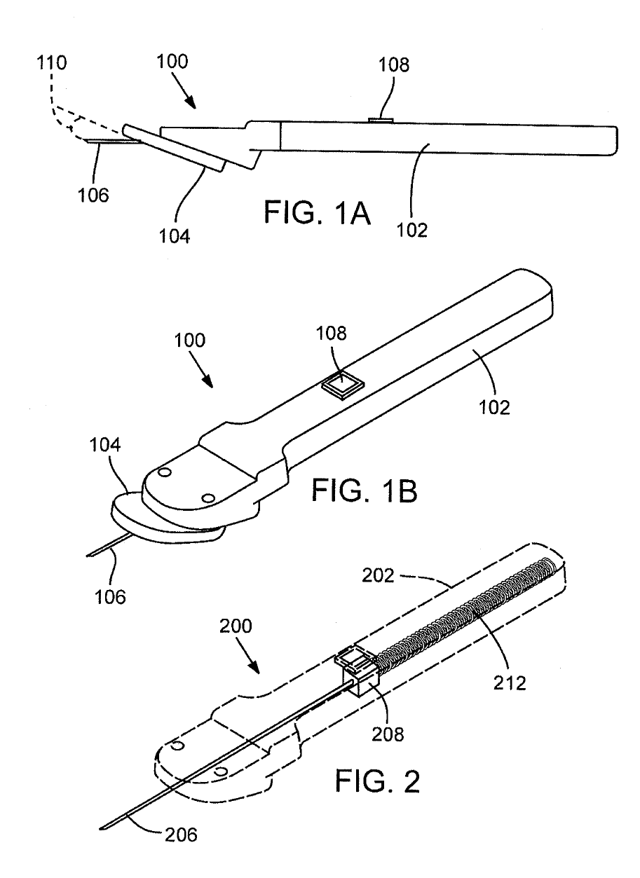

FIGS. 1A and 1B illustrate an embodiment of the present invention in which a transdermal or transcutaneous introducer assembly 100 includes a handle 102, a module base 104 coupled to handle 102, and a hollow introducer 106, supported by and extending from handle 102 and passing through and protruding from module base 104. Prior to and during insertion into a body, a transcutaneous sensor (not shown) may reside in the hollow space of introducer 106. In an embodiment, activation of button 108 may cause introducer 106 to retract, and, in an embodiment, after retraction, introducer 106 may be fully contained within an interior volume of handle 102. In an embodiment, activation of a button 108 may cause handle 102 to release module base 104, directly or via interaction with the retracting introducer.

Handles in accordance with embodiments of the present invention may have a variety of sizes and shapes. In embodiments, a handle may be straight, squared, curved, etc., may have a variety of lengths and/or widths, and may, in embodiments, be designed for easy and comfortable use by a user.

A module base in accordance with an embodiment of the present invention may include an electrical network adapted to receive an electrical signal from an associated sensor and to transmit a further signal, for example to an external electronic monitoring unit, that is responsive to the sensor signal. In embodiments, an electrical network may comprise a variety of components in any desired structural relationship, whether or not the network has a printed circuit board, a tethered or wired system, etc. In an embodiment, signal transmission may occur over the air with electromagnetic waves, such as RF communication, or data may be read using inductive coupling. In other embodiments, transmission may be over a wire or via another direct connection.

In an embodiment of the present invention, additional components may be housed in one or more separate modules that may be coupled to (for example, snapped to, wired to, or in wireless communication with) the module base. For example, the separate module may contain a memory component, a battery component, a transmitter, a receiver, a transceiver, a processor, and/or a display component, etc.

In an embodiment, the module base and associated sensor may be affirmatively linked together. When the sensor is withdrawn, so is the module base and they are both disposed of together. In an alternative embodiment, the module base stays on the patient during the lifetime of a sequence of transcutaneous sensors. In such an embodiment, the module base and the sensor are constructed so as to be quickly and easily connected and disconnected. For example, in an embodiment, the sensor and module base may be provided with easily connected and disconnected male and female pins, respectively.

In an embodiment, an introducer assembly may introduce a sensor for the sensing of glucose or lactate or other analytes of interest.

FIGS. 1A and 1B show button 108, however, a button is merely an exemplary embodiment of a variety of actuators for retraction mechanisms, such as a button or a pin, whether depressable, retractable, pullable, rotatable, slidable, etc. In an embodiment such as shown in FIG. 2, a retraction mechanism 208 includes a spring 212, for example a tension spring in the tensed (extended) position. Upon actuation, spring 212 may be allowed to return to a relaxed state retracting introducer 206 into handle 202.

To use, referring again to FIGS. 1A and 1B, assembly 100 may be brought toward the skin of an animal, typically a human, and introducer 106 may be pushed through the skin into the individual's subcutaneous tissue. In an embodiment, the sole motive force of entry is provided by the user, however, in an alternative embodiment, a separate motive force, such as a spring, lever, etc. may be provided to drive the introducer into the individual's skin. The angle of entry of the introducer into the skin is defined by two main factors: the predetermined exit angle 110 between introducer 106 and the lower surface of module 104, and the approach angle of the user. In an embodiment, exit angle 110 may be between 0° and 90°, for example 0°-45°, for example approximately 15-25°. In operation, a sensor resides in introducer 106 and is thus inserted into the individual along with introducer 106.

In embodiments, prior to insertion, a sensor may be contained entirely within an introducer or may extend slightly beyond the distal tip of the introducer. In an embodiment, button 108 may be actuated and handle 102 and introducer 106 may be withdrawn, leaving the sensor (not shown) in the tissue of the animal (again, typically a human patient) and coupled in some manner to module base 104 on the skin.

FIG. 2 shows a cross-section of an assembly 200 having a spring 212 inside handle 202 and a retraction mechanism 208 coupled to an introducer 206. The manner in which retraction mechanism 208 communicates with spring 212 depends on the particular type of retraction mechanism utilized, whether it is a button, pin, etc. and such interaction is within the knowledge of one of ordinary skill in light of the teachings of the present specification. In an embodiment, a retraction mechanism may include an actuator (button, pin, etc.) coupled to a motive force device (spring, lever, etc.).

U.S. Pat. No. 6,695,860 provides an exemplary embodiment for retraction of an introducer after implantation of a sensor using a tension spring and a retraction actuating pin. The entire contents and disclosure of U.S. Pat. No. 6,695,860 is hereby incorporated by reference.

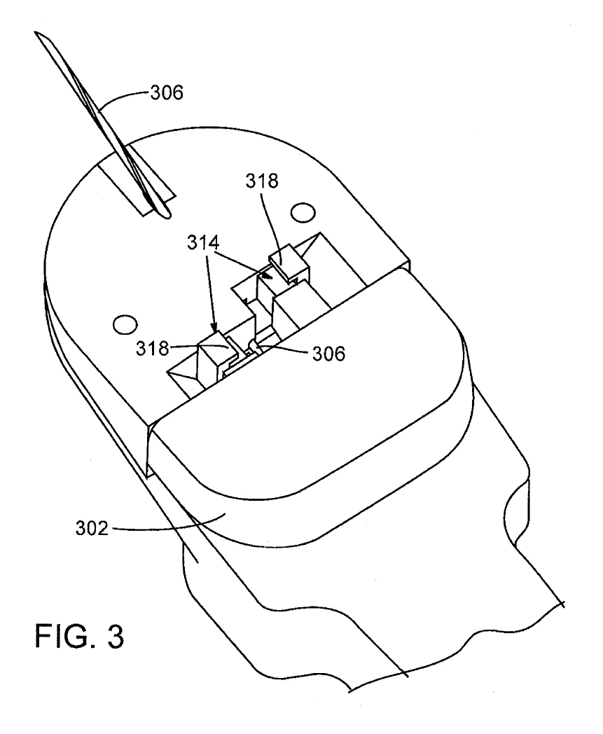

FIG. 3 shows a partial cross-sectional view (from the underside, module-interacting side) of handle 302 in which introducer 306 resides, and from which introducer 306 extends. When introducer 306 is extended (as shown in FIG. 3), clips 314 are biased against introducer 306 by force provided by springs (not shown), such as compression springs. In an embodiment, introducer 306 is not held firmly in place, but rather is slidably engaged with clips 314. In addition, clips 314 have connector tabs 318 which interact with corresponding indentations in an associated module base (not shown). When introducer 306 is retracted into handle 302 past the region of clips 314, the springs force clips 314 to pivot releasing connector tabs 318 from interaction with the module base and thus releasing handle 302 from the module base.

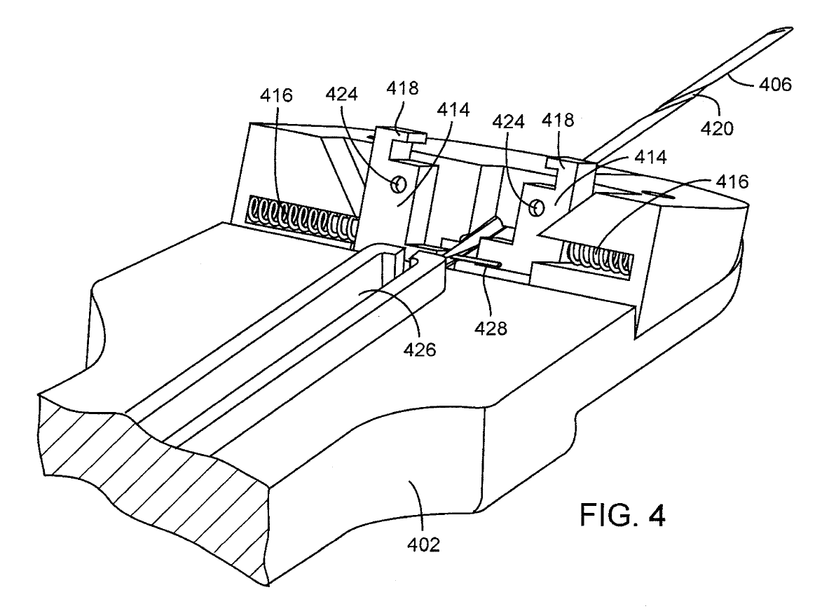

FIG. 4 shows a detailed partial cross-sectional view of a portion of a handle 402 and the mechanics of clips 414. Clips 414 have connector tabs 418 which interact with corresponding indentations in an associated module base (not shown). When introducer 406 is extended, clips 414 are biased against introducer 406 by force provided by springs 416, such as compression springs. Furthermore, when introducer 406 is retracted into handle 402 past the region of clips 414, springs 416 force clips 414 to pivot around pivot points 424 opening the space between connector tabs 418 and releasing connector tabs 418 from interaction with the module base and thus releasing handle 402 from the module base. In an embodiment, when introducer 406 is retracted, introducer 406 may reside in retraction channel 426.

As shown in FIG. 4, such an embodiment may have an introducer 406 with a curved slot 420 and a pin 428 that cooperates with slot 420. In an embodiment of the present invention, a pin may be provided on the module base or in the handle, and may be in a variety of locations to cooperate with introducer slots. In an embodiment such as shown in FIG. 4, in operation, as introducer 406 is retracted, introducer 406 may be rotated about pin 428 so that the sensor (not shown) may easily slip out of introducer 406 as introducer 406 is being withdrawn. In other words, pin 428 orients slot 420 in a desired position to allow the sensor to be aligned with slot 420 for removal of the sensor and withdrawal of introducer 406.

In embodiments, a slot in an introducer begins at the distal tip of the introducer and extends at least partially along the length of the introducer. In embodiments, the slot defined by an introducing introducer may be straight and the sensor may be retained within it during insertion because of the stiffness or rigidity of the sensor or because the slot is relatively narrow and the sensor cannot easily pass through it. In an embodiment, a slot may extend along the entire length of the introducer or may extend along only a portion of the introducer.

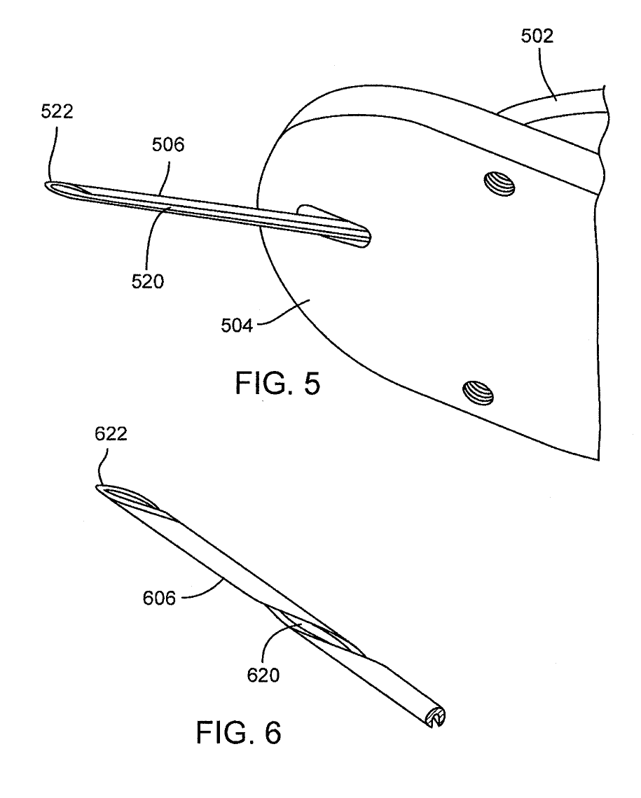

FIG. 5 shows the underside (skin side) of module base 504 coupled to handle 502 and with an extended introducer 506. Introducer 506 has a straight slot 520 originating at distal tip 522 of introducer 506 and extending along introducer 506. In an embodiment, introducer 506 extends from handle 502 and passes through module base 504 with minimal interaction with module base 504, except primarily to guide and insert an associated sensor that is, for example, coupled to module base 504.

U.S. Pat. No. 7,003,336, the entire contents and disclosure of which is hereby incorporated by reference, shows additional features for use with embodiments of the present invention in which a device possesses an introducer for introducing a sensor. The introducer has a straight slot extending along a portion of the introducer to allow for withdrawal of the introducer after introduction of the sensor. In addition, a notched sensor is provided such that the notched portion can slide through the slot, but the remaining portions of the sensor cannot pass through the slot.

In an embodiment of the present invention, a sensor with substantially uniform cross-section may be utilized (i.e., without a notch in the sensor) based on various arrangements of introducers and associated slots described herein. Alternatively, in an embodiment of the present invention, a sensor with a varied cross section may be used. In embodiments, a sensor may be cylindrical, squared, rectangular, etc. In an embodiment, a sensor a may be used having a cross-sectional width that is less than the width of the slot. In an embodiment, a sensor may be a wire-type sensor. In an embodiment, a sensor may be flexible.

In an embodiment of the present invention, the slot defined by an introducing introducer may be curved so as to more successfully retain a sensor, such as a flexible sensor, that might slip out of a straight slot. A curved slot may be uniform or regular along the portion of the introducing introducer in which the slot is present, or the curve may be irregular. For example, a curved slot need not continue around an introducer, but in an embodiment may start in one axial direction and then turn in a different direction along the introducer.

In an embodiment, a curved slot may be helical in shape. For the purposes of the present invention, the term “helical” refers to a curve that is spirally coiled about an axis.

FIG. 6 shows an introducer 606 with a helical slot 620 originating at distal tip 622 of introducer 606 and extending along and curving around introducer 606.

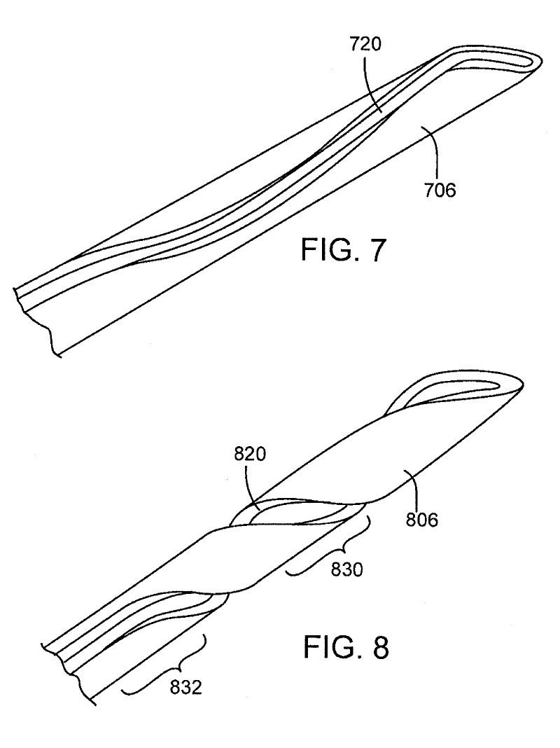

FIG. 7 shows an exemplary curved slot 720 in an introducer 706 according to an embodiment of the present invention.

In an embodiment, a portion of an introducer may have a curved slot located at the distal tip of the introducer followed by and adjacent to a more proximal portion of the introducer in which the slot is straight. Such a slot may continue along the entire introducer, or may only be present for a portion of the introducer. In an embodiment, a curved slot extends along an introducer for a portion of the introducer at least overlapping the distal tip of the sensor in a position prior to insertion. Such an embodiment helps retain the sensor in the introducer prior to and during insertion.

An exemplary embodiment showing introducer 806 and various regions of slot 820, including a distal curved portion 830 and an adjacent straight portion 832, is shown in FIG. 8.

Thus, in an embodiment of the present invention there is provided an introducer for introducing an analyte sensor into a body, having an elongated hollow shaft having at least a distal segment beginning at the distal tip of the shaft and a proximal segment adjacent to the distal segment; and a longitudinal curved slot extending along at least a portion of the shaft.

In an embodiment, a slot may be weakly sealed so that the seal is broken as the sensor is withdrawn. A weak seal may be provided by wax, a thin membrane, foil, etc. which may be broken during withdrawal of the introducer. The seal provides an additional mechanism to maintain the sensor in the interior hollow portion of the introducer. A pin, as discussed above, in interaction with the slot, may be used to break the seal during withdrawal of the introducer.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A transdermal introducer assembly, comprising:
a module base adapted for mounting onto an animal's skin, and supporting an electrical network;
a handle defining an interior volume and configured for removable coupling with said module base;
a hollow introducer, having a longitudinal slot extending at least partially along the length of said hollow introducer, said hollow introducer carried by and extending from said handle in a predetermined orientation; and
a retraction mechanism associated with said handle and configured to retract said introducer into said interior volume of said handle and to permit removal of said handle from said module base.

2. The assembly of claim 1, further comprising a sensor having a proximal segment electrically coupled to said module base, and a distal segment protruding from said module base.

3. The assembly of claim 2, wherein said distal segment of said sensor resides at least partially in said introducer.

4. The assembly of claim 3, wherein said slot is curved and said introducer communicates with a pin, and, upon retraction, said introducer rotates about said pin along said curved slot to permit disengagement of said introducer from said sensor.

5. The assembly of claim 3, wherein said slot is covered with a weak sealing material to retain said sensor during insertion of said introducer and said sensor and to permit escape of said sensor from said introducer during retraction of said introducer by breaking said weak sealing material.

6. The assembly of claim 5, wherein said weak sealing material comprises a thin membrane, wax, or foil.

7. The assembly of claim 2, wherein said sensor is flexible.

8. The assembly of claim 1, wherein said slot extends along the entire length of said introducer.

9. The assembly of claim 1, wherein said slot is curved.

10. The assembly of claim 9, wherein said curved slot is helical.

11. The assembly of claim 1, wherein said retraction mechanism comprises a button or a pin coupled to a spring, and wherein said spring is further coupled to said introducer.

12. The assembly of claim 1, further comprising spring actuated clips mounted in said handle for slidable engagement with said introducer when said introducer is in an extended position.

13. The assembly of claim 12, wherein said spring actuated clips further comprise connector tabs for removably coupling said handle and said module base.

14. The assembly of claim 1, wherein said longitudinal slot extends from the distal tip of said introducer along a first segment of said introducer as a curved slot and wherein said curved slot transitions to a straight slot extending along at least a second segment of said introducer adjacent to said first segment.

15. An introducer for introducing an analyte sensor into a body, comprising:
an elongated hollow shaft having at least a distal segment beginning at the distal tip of said shaft and a proximal segment adjacent to said distal segment; and
a longitudinal curved slot extending along at least a portion of said shaft.

16. The introducer of claim 15, wherein said curved slot is helical.

17. The introducer of claim 15, wherein said curved slot extends from the distal tip of said shaft along at least said distal segment.

18. The introducer of claim 15, wherein said curved slot extends from the distal tip of said shaft along said distal segment and wherein said curved slot transitions to a straight slot extending along at least said proximal segment.

19. A method for inserting an analyte sensor into an animal's tissue, comprising:
inserting a hollow introducer into an animal's tissue, said introducer being part of an introducer assembly comprising:
a module base adapted for mounting onto an animal's skin, and supporting an electrical network;
a handle defining an interior volume and configured for removable coupling with said module base;
a hollow introducer, having a longitudinal slot extending at least partially along the length of said hollow introducer, said hollow introducer carried by and extending from said handle in a predetermined orientation;
a sensor residing at least partially in said introducer and having a proximal segment electrically coupled to said module base, and a distal segment protruding from said module base; and
a retraction mechanism associated with said handle and configured to retract said introducer into said interior volume of said handle and to permit removal of said handle from said module base; and
activating said retraction mechanism to retract said introducer into said handle and to disengage said handle from said module base, while leaving said sensor in said animal tissue.

20. The method of claim 1, wherein said handle further comprises a retraction channel, and wherein, upon retraction, said introducer is retracted completely into said retraction channel in said handle.
